(19) United States
(12) Patent Application Publication
(10) Pub. No.: US 2019/0183461 A1
(43) Pub. Date: Jun. 20, 2019

(54) ULTRASONIC DIAGNOSTIC DEVICE

(71) Applicant: HITACHI, LTD., Tokyo (JP)

(72) Inventor: Teruyuki SONOYAMA, Chiyoda-ku, Tokyo (JP)

(73) Assignee: HITACHI, LTD., Tokyo (JP)

(21) Appl. No.: 16/326,676

(22) PCT Filed: Aug. 2, 2017
(86) PCT No.: PCT/JP2017/027995

§ 371 Date: Feb. 19, 2019

(30) Foreign Application Priority Data
Aug. 25, 2016 (JP) 2016-164306

(51) Int. Cl. A61B 8/08; A61B 8/00

(52) U.S. Cl. CPC A61B 8/485; A61B 8/469; A61B 8/463; A61B 8/5292

(57) ABSTRACT

Strain elastography measurement is performed based on data in a first region of interest, and immediately, shear wave elastography measurement is performed based on data in a second region of interest. The first region of interest and the second region of interest are set with a region of interest setting unit in correspondence with a user operation. A display image forming unit forms a first marker R1 corresponding to the first region of interest in a strain elastography image 84A, in the first-performed strain elastography measurement, and further, forms a second marker R2 corresponding to the second region of interest and a reference marker RM corresponding to the first region of interest in a shear wave elastography image 84B.

### TECHNICAL FIELD

The present invention relates to an ultrasonic diagnostic device, and more particularly, to a device to perform elasticity measurement utilizing an ultrasonic wave.

### BACKGROUND ART

In an ultrasonic diagnostic device, a technique of obtaining diagnostic information related to tissue elasticity is known. For example, strain elastography (strain elastography) for obtaining diagnostic information related to tissue elasticity is known as a method of pressing the tissue in a subject from the body surface of the subject and measuring a strain of the tissue caused by the pressing with an ultrasonic wave.

Further, shear wave elastography (shear wave elastography) is known as a method of generating a shear wave (shear wave) in the subject with an ultrasonic wave pushing pulse, and obtaining diagnostic information related to tissue elasticity from the speed of the shear wave propagated in the tissue.

For example, patent literatures 1 and 2 disclose an ultrasonic diagnostic device provided with both strain elastography and shear wave elastography functions.

### CITATION LIST

### Patent Literature

Patent Literature 1: Japanese Patent No. 5559788

Patent Literature 2: Japanese Patent No. 5848793

### SUMMARY OF INVENTION

### Technical Problem

The both methods of strain elastography and shear wave elastography respectively have advantages and disadvantages. Accordingly, with an ultrasonic diagnostic device having the functions of the both elastography functions, it is possible to perform measurement with e.g. the advantages of the both methods.

However, there is a difference of measurement method between both of the strain elastography and shear wave elastography, and there is a difference of function setting and the like required for measurement between them. Accordingly, e.g., when elasticity measurement is performed on the same region by both methods of the strain elastography and shear wave elastography, it is not easy to optimize the measurement conditions of the both methods so as to perform equivalent measurement in the both methods.

The present invention has an object to provide a technique of optimizing measurement conditions of plural methods in elasticity measurement utilizing an ultrasonic wave.

### Solution to Problem

A preferred ultrasonic diagnostic device to attain the above-described object is an ultrasonic diagnostic device comprising: an elasticity measurement unit that performs elasticity measurement according to a first method and elasticity measurement according to a second method based on data obtained by transmission/reception of an ultrasonic wave; a region of interest setting unit that sets a first region of interest corresponding to the elasticity measurement according to the first method and a second region of interest corresponding to the elasticity measurement according to the second method; and a display image forming unit that forms a first display image corresponding to the elasticity measurement according to the first method and a second display image corresponding to the elasticity measurement according to the second method, wherein the elasticity measurement unit performs the elasticity measurement according to the first method based on data in the first region of interest in the elasticity measurement according to the first method, and wherein the display image forming unit forms a display image including the first display image and the second display image, forms a first marker corresponding to the first region of interest in the first display image, and forms a second marker corresponding to the second region of interest in the second display image, in the elasticity measurement according to the first method, further wherein the elasticity measurement unit performs the elasticity measurement according to the second method based on data in the second region of interest in the elasticity measurement according to the second method after the elasticity measurement according to the first method.

According to the above-described configuration, in the elasticity measurement according to the first method, since the first marker corresponding to the first region of interest and the second marker corresponding to the second region of interest are formed in the display image, it is possible to adjust the positional relationship or the like between the first region of interest and the second region of interest, thus perform optimization before the elasticity measurement according to the second method. It is desirable that the adjustment is performed before determination of measurement result from the elasticity measurement according to the first method. When the measurement result from the elasticity measurement according to the first method is not determined, it is possible to optimize the position or the like of the first region of interest utilized in the first method even in consideration of the second region of interest to be utilized later in the second method.

In the preferred concrete example, the display image forming unit forms a reference marker corresponding to the first region of interest, together with the second marker, in the second display image, in the elasticity measurement according to the first method.

In the preferred concrete example, the region of interest setting unit has an interlocking setting function of changing a setting position of one of the first region of interest and the second region of interest in accordance with change of the setting position of the other one.

In the preferred concrete example, the elasticity measurement unit performs strain elastography measurement based on displacement distribution of a tissue in a living body, as the elasticity measurement according to the first method, and performs shear wave elastography measurement based on a shear wave propagated through the tissue in the living body, as the elasticity measurement according to the second method.

In the preferred concrete example, the ultrasonic diagnostic device obtains a general diagnostic result of the tissue in the living body based on a measurement result obtained from the tissue in the living body by the strain elastography measurement and a measurement result obtained from the tissue in the living body by the shear wave elastography measurement. Further, in the preferred concrete example, the ultrasonic diagnostic device obtains a general diagnostic result of the tissue in the living body based on a measurement result obtained from the tissue in the living body by the strain elastography measurement, a measurement result obtained from the tissue in the living body by the shear wave elastography measurement, and blood data of the subject having the tissue in the living body.

### Advantageous Effects of Invention

In accordance with the present invention, in elasticity measurement utilizing an ultrasonic wave, a technique of optimizing measurement conditions in plural methods is provided. For example, according to a preferred aspect of the present invention, before the elasticity measurement according to the second method, or desirably before determination of the measurement result from the elasticity measurement according to the first method, it is possible to optimize the positional relationship or the like between the first region of interest and the second region of interest.

### BRIEF DESCRIPTION OF DRAWINGS

FIG. 1 is a diagram showing an entire configuration of a preferred ultrasonic diagnostic device according to an embodiment of the present invention.

FIG. 2 is a diagram for explaining a concrete example of strain elastography measurement.

FIG. 3 is a diagram for explaining a concrete example of shear wave elastography measurement.

FIG. 4 is a diagram showing a concrete example of measurement result at a propagation velocity Vs.

FIG. 5 is a diagram showing a concrete example of a display image in combinational elastography.

FIG. 6 is a diagram showing a concrete example of blood data.

### DESCRIPTION OF EMBODIMENT

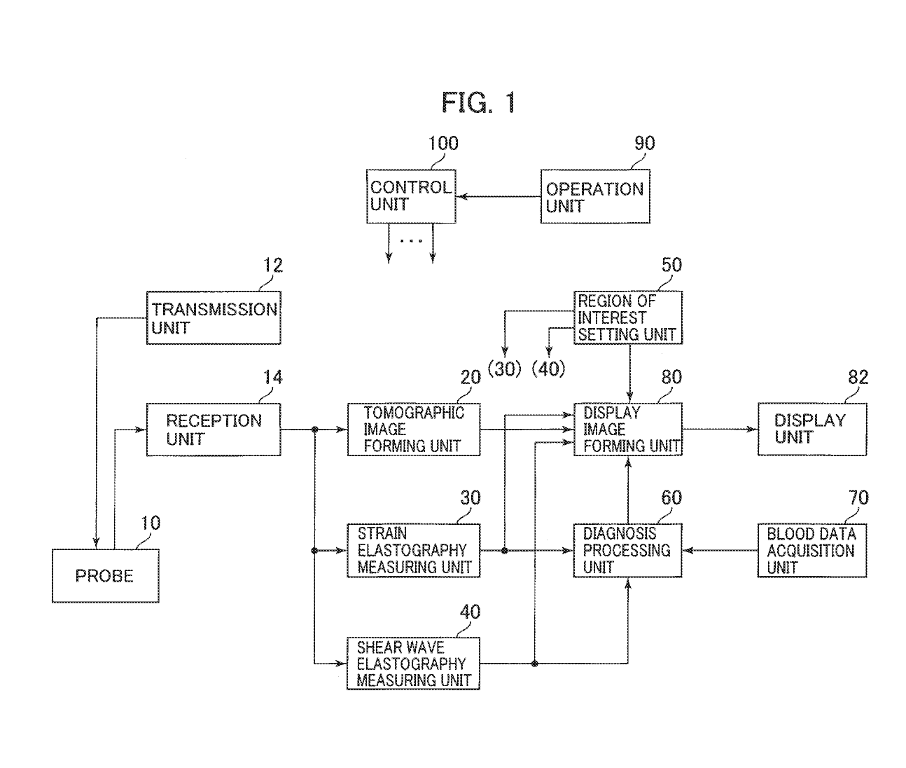

FIG. 1 is a diagram showing an entire configuration of a preferred ultrasonic diagnostic device according to an embodiment of the present invention. A probe 10 is an ultrasonic probe to transmit/receive an ultrasonic wave with respect to a region including a diagnostic object such as a tissue or the like in a subject (living body). The probe 10 has plural vibration elements respectively for transmission/reception or wave transmission. The plural vibration elements are transmission-controlled with a transmission unit 12, thus a transmission beam is formed.

Further, the plural vibration elements of the probe 10 receive an ultrasonic wave from the region including the diagnostic object, and a signal obtained by the wave reception is outputted to a reception unit 14. The reception unit 14 forms a reception beam, and along the reception beam, a reception signal (echo data) is collected. Note that the probe 10 may desirably be a convex type probe but may be a linear type probe or the like.

The probe 10 has a function of transmitting/receiving an ultrasonic wave (normal pulse) to obtain frame data from a cross section in the subject, a function of transmitting an ultrasonic wave (pushing pulse) to generate a shear wave in the subject, and a function of transmitting/receiving an ultrasonic wave (tracking pulse) for measurement of the shear wave.

A tomographic image forming unit 20 forms ultrasonic-wave tomographic image data based on multi-time-phase frame data obtained from the reception unit 14. The tomographic image forming unit 20 performs signal processing such as gain correction, log compression, detection, contour enhancement, and filter processing, on the frame data formed with the reception data, in accordance with necessity, so as to form e.g. image data of a B-mode image in the cross section in the subject, as tomographic image data. Note that it is desirable that the tomographic image forming unit 20 has a frame memory capable of storing multi-time-phase (plural frames) tomographic image data.

A strain elastography measuring unit 30 performs well-known strain elastography (strain elastography) based on displacement distribution of a tissue in the subject (in the living body). A shear wave elastography measuring unit 40 performs well-known shear wave elastography (shear wave elastography) based on the shear wave propagated in the subject (in the living body). Further, a region of interest setting unit 50 sets a first region of interest utilized in strain elastography measurement and a second region of interest utilized in shear wave elastography measurement.

A diagnosis processing unit 60 obtains a general diagnostic result of the tissue in the subject, based on a measurement result obtained from the subject by the strain elastography measurement, and a measurement result obtained from the subject by the shear wave elastography measurement. Upon acquisition of the diagnostic results, the diagnosis processing unit 60 utilizes blood data obtained from a blood data acquisition unit 70.

A display image forming unit 80 forms a display image based on the tomographic image data obtained from the tomographic image forming unit 20, or the like. The display image formed with the display image forming unit 80 is displayed on a display unit 82.

A control unit 100 performs overall control in the ultrasonic diagnostic device in FIG. 1. The overall control with the control unit 100 reflects an instruction received via an operation unit 90 from a user such as a doctor or a laboratory technologist.

Among the constituent elements (respective units with reference numerals) shown in FIG. 1, the transmission unit 12, the reception unit 14, the tomographic image forming unit 20, the strain elastography measuring unit 30, the shear wave elastography measuring unit 40, the region of interest setting unit 50, the diagnosis processing unit 60, the blood data acquisition unit 70, and the display image forming unit 80 may be respectively realized by utilizing hardware such as electrical and electronic circuits and processors. Upon implementation of these units, a device such as a memory may be utilized if necessary. Further, at least a part of the functions corresponding to the above-described units may be realized with a computer. That is, at least a part of the functions corresponding to the above-described units may be realized by cooperation between hardware such as a CPU, a processor or a memory and software (program) to prescribe the operation of the CPU, the processor or the like.

A preferred concrete example of the display unit 82 is a liquid crystal display, an organic EL (Electro-Luminescence) display or the like. The operation unit 90 may be realized with at least one of e.g. a mouse, a keyboard, a track ball, a touch panel, and other switches. The control unit 100 may be realized by cooperation between hardware such as a CPU, a processor or a memory, and software (program) to prescribe the operation of the CPU, the processor or the like.

The entire configuration of the ultrasonic diagnostic device in FIG. 1 is as described above. Next, the functions and the like related to the elasticity measurement realized with the ultrasonic diagnostic device in FIG. 1 will be described in detail. Note that regarding the constituent elements (parts) shown in FIG. 1, the reference numerals in FIG. 1 will be used in the following description.

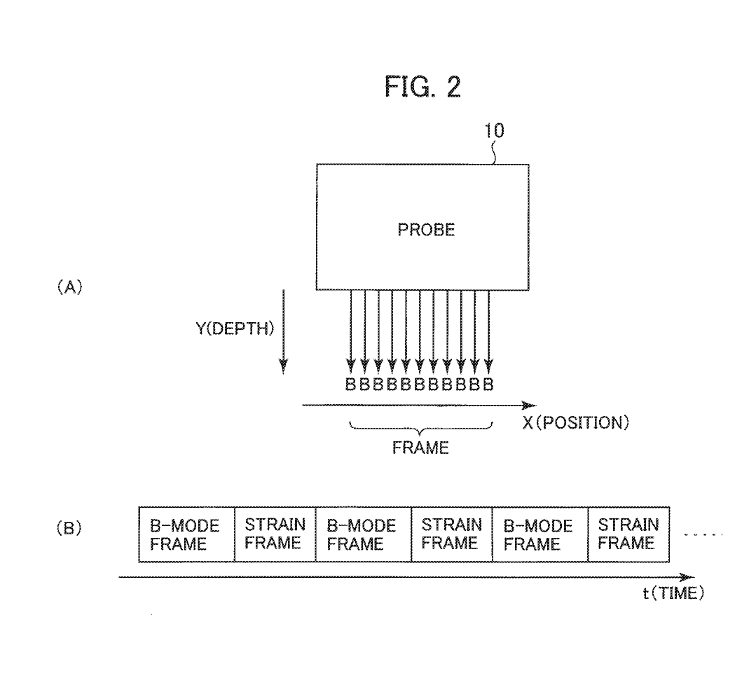

FIG. 2 is a diagram for explaining a concrete example of the strain elastography measurement. The strain elastography measuring unit 30 performs well-known strain elastography (strain elastography) based on the displacement distribution of the tissue in the subject (in the living body). The displacement distribution of the tissue in the subject (in the living body) is derived based on the frame data. The frame data is formed by scanning an ultrasonic beam B in the cross section of the subject.

FIG. 2(A) shows a concrete example of the ultrasonic beam B scanned in the subject by utilizing the probe 10. When the frame data is obtained, the transmission unit 12 outputs a normal pulse transmission signal to the plural vibration elements of the probe 10, to transmission-control the probe 10 so as to form a normal pulse transmission beam and scan the transmission beam. Further, the reception unit 14 performs phasing addition processing or the like on the received signal obtained from the plural vibration elements by transmission/reception of the normal pulse ultrasonic wave with the probe 10, so as to form a reception beam corresponding to the normal pulse transmission beam, and obtain reception data (e.g. RF signal data) along the reception beam.

The normal-pulse ultrasonic beam B (transmission beam and reception beam) is scanned in the cross section of the subject, and the frame data is formed with the reception data collected from the cross section. For example, as shown in FIG. 2(A), the ultrasonic beam B is sequentially formed along a depth Y direction while the position on an X-axis is shifted, thus 1-frame frame data is obtained. The frame data is formed by time phase over the multiple time phases, i.e., by frame over the plural frames.

FIG. 2(B) shows a concrete example of the frame data sequentially obtained over the multiple time phases in the strain elastography measurement. In FIG. 2(B), a lateral axis indicates a time axis t. In the strain elastography measurement, as in the case of e.g. the concrete example shown in FIG. 2(B), frame data for B-mode image (B-mode frame) and frame data for strain elastography measurement (strain frame) are alternately formed.

The tomographic image forming unit 20 forms ultrasonic-wave tomographic image data dynamically projecting, e.g., the tissue in the subject based on the B-mode frame frame data sequentially obtained by time phase over multiple time phases.

The strain elastography measuring unit 30 performs the well-known strain elastography measurement based on strain-frame frame data sequentially obtained by time phase over the multiple time phases. In the strain elastography, e.g. the probe 10 is applied to the body surface of the subject, then the tissue in the subject is pressed from the body surface of the subject, and the displacement of the tissue with the pressing is measured.

The strain elastography measuring unit 30 measures the displacement of the tissue in the subject based on frame data of a set of strain frames, selected by time phase over the multiple time phases, e.g. two strain frames corresponding to mutually adjacent time phases.

The strain elastography measuring unit 30 performs e.g. one-dimensional or two-dimensional correlation operation processing on the one set of strain frames, and by measurement point in the frame data i.e. the tomographic image, derives a displacement vector indicating the displacement of the tissue at that measurement point, i.e. a one-dimensional or two-dimensional displacement vector related to the direction and magnitude of the displacement. Thus the strain elastography measuring unit 30 obtains the distribution (displacement distribution) of the displacement vectors in the plural measurement points in the tomographic image. Upon derivation of the displacement vector, e.g., well-known block matching or phase gradient method is used.

Further, the strain elastography measuring unit 30 obtains elasticity information of the tissue in the subject based on the displacement distribution of the tissue in the subject. The strain elastography measuring unit 30 calculates the strain or the modulus of the tissue by measurement point of the plural measurement points based on the displacement vector at each measurement point, measured between e.g. a set of strain frames. Further, the strain elastography measuring unit 30 calculates the strain or the elastic modulus of the tissue at the plural measurement points, by time phase (each strain frame) over the multiple time phases.

Then the strain elastography measuring unit 30 forms an elasticity image to visually indicate the elasticity information in the cross section of the subject based on the elasticity frame data indicating the elasticity information (strain or elastic modulus of the tissue) at each measurement points at each time phase (in each strain frame). Also a well-known method is utilized upon formation of the elasticity image. The strain elastography measuring unit 30 has a function of providing hue information corresponding to elasticity information at each measurement point, to the respective measurement points of e.g. elasticity frame data. The strain elastography measuring unit 30 forms elasticity image data, in which red (R), green (G), and blue (B) as light three primary colors are provided to each measurement point, based on the elasticity frame data. For example, red-based hue information is provided to elasticity data with high strain and blue-based hue information is provided to elasticity data with low strain. Thus elasticity image data is formed by time phase (by frame) over the multiple time phases.

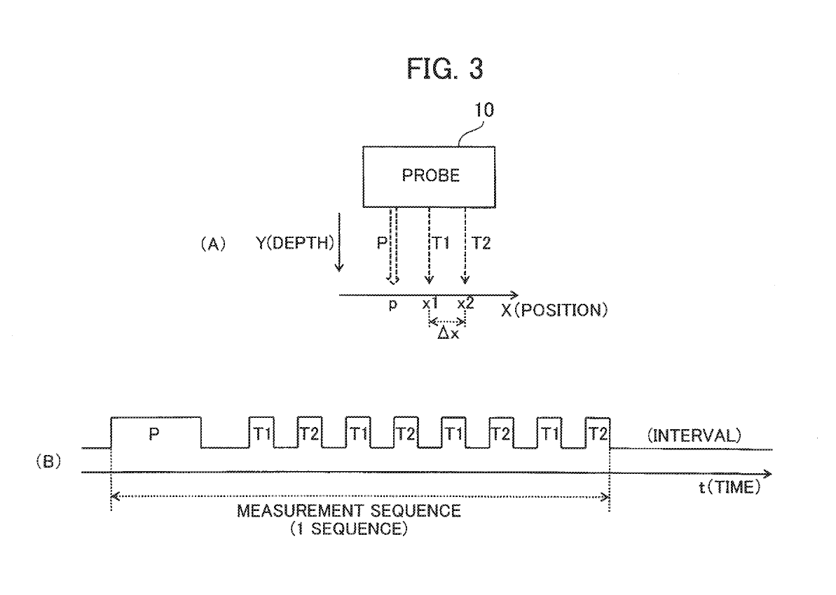

FIG. 3 is a diagram for explaining a concrete example of the shear wave elastography measurement. Utilizing FIG. 3, the concrete example related to measurement of the occurrence and displacement of a shear wave in the shear wave elastography (shear wave elastography) will be described.

To cause a shear wave, the transmission unit 12 outputs a pushing pulse (pushing wave) transmission signal to the plural vibration elements of the probe 10, to transmission-control the probe 10 so as to form a pushing pulse transmission beam. Further, to measure the shear wave, the transmission unit 12 outputs a tracking pulse (tracking wave) transmission signal to the plural vibration elements of the probe 10, to transmission-control the probe 10 so as to form a tracking pulse transmission beam. Then the reception unit 14 performs phasing addition processing or the like on a reception signal, obtained from the plural vibration elements by transmission/reception of the tracking pulse with the probe 10, to form a tracking pulse reception beam, and obtain reception data (e.g. RF signal data) along the reception beam.

FIG. 3(A) shows a concrete example of a pushing wave transmission beam P formed by utilizing the probe 10, and tracking wave ultrasonic beams T1 and T2. In the concrete example shown in FIG. 3(A), the pushing wave transmission beam P is formed along the depth Y direction so as to pass through an X direction position p. For example, the pushing wave transmission beam P is formed with the position p on the X-axis shown in FIG. 3(A) as a focal point. The position p is set by e.g. a user (inspector) such as a doctor or a laboratory technologist who checked an ultrasonic wave image (tomographic image formed with the tomographic image forming unit 20) related to a diagnostic object in the living body displayed on the display unit 82, to a desired position.

When the transmission beam P is formed with the position p as a focal point and the pushing wave is transmitted, then in the living body, a comparatively strong shear wave occurs in the position p and its peripheral part. FIG. 3(A) shows a concrete example of measurement of propagation velocity in the shear X direction occurred in the position p.

In the concrete example of FIG. 3(A), the two tracking wave ultrasonic beams T1 and T2 are formed. The ultrasonic beam (transmission beam and reception beam) T1 is formed so as to pass through e.g. a position x1 on the X-axis shown in. FIG. 3(A). The ultrasonic beam (transmission beam and reception beam) T2 is formed so as to pass through e.g. a position x2 on the X-axis shown in FIG. 3(A). The position x1 and the position x2 may be set by e.g. the user who checked the ultrasonic wave image of the diagnostic object displayed on the display unit 82, to desired positions. Otherwise, the position x1 and the position x2 may be set to a place away from the position p by a predetermined distance along the X direction.

Note that in the concrete example shown in FIG. 3(A), the tracking wave ultrasonic beams T1 and T2 are formed on the X-axis positive direction side with respect to the pushing wave transmission beam P. It may be configured such that the tracking wave ultrasonic beams T1 and T2 are formed in the X-axis negative direction side with respect to the pushing wave transmission beam P and the shear wave propagated on the X-axis negative direction side is measured. It goes without saying that it is desirable that the position p of the pushing wave transmission beam P and the positions x1 and x2 of the tracking wave ultrasonic beams T1 and T2 are appropriately set in correspondence with diagnostic object, diagnostic situation and the like.

FIG. 3(B) shows a concrete example of generation timings of the pushing wave transmission beam P, and the tracking wave ultrasonic beams T1 and T2. The lateral axis in FIG. 3(B) is the time axis t.

In FIG. 3(B), a period P is a period where the pushing wave transmission beam P is formed. Periods T1 and T2 are respective periods where the tracking wave ultrasonic beams T1 and T2 are formed.

In the period P, a multiple wave pushing wave is transmitted. For example, a continuous wave ultrasonic wave is transmitted in the period P. Then, e.g., from immediately after the end of the period P, a shear wave occurs in the position p.

In the periods T1 and T2, about one to several waves of so-called pulse wave tracking waves are transmitted, and reflected waves accompanying the pulse waves are received. For example, the ultrasonic beams T1 and T2 passing through positions x1 and x2 are formed, and reception signals are obtained in plural depths including the positions x1 and x2. That is, regarding the respective ultrasonic beams T1 and T2, reception signals are obtained from plural depths.

The transmission/reception of the tracking wave is repeated over plural periods. For example, as shown in FIG. 3(B), the periods T1 and T2 are alternately repeated until e.g. the displacement of the tissue accompanying the shear wave has been checked.

The shear wave elastography measuring unit 40 calculates a propagation velocity Vs of the shear wave in the X-axis direction based on the displacement in the positions x1 and x2 varied due to the influence of the shear wave occurred in the position P. For example, the propagation velocity Vs=Δx(t2−t1) of the shear wave in the X-axis direction is calculated based on time t1 at which the displacement in the position x1 is maximum, time t2 at which the displacement in the position x2 is maximum, and a distance Δx between the position x1 and the position x2. Note that the propagation velocity of the shear wave may be calculated by utilizing other well-known methods.

Further, the shear wave elastography measuring unit 40 calculates the propagation velocity Vs by each of the plural depths based on e.g. the reception signals obtained from the ultrasonic beam T1 and the ultrasonic beam T2. Further, the elasticity information such as the elasticity value of a tissue, where the shear wave is measured, may be calculated based on the propagation velocity Vs of the shear wave. Otherwise, as the tissue information, a viscoelasticity parameter, attenuation, a frequency characteristic and the like, may be derived.

A measurement sequence shown in FIG. 3(B) is a period of one sequence from the start of pushing wave transmission to the calculation of the propagation velocity of the shear wave. It is desirable that for cleaning of the probe 10, an interval is provided after the end of the measurement sequence. Further, after the interval, the next measurement sequence is started, thus the plural measurement sequences are repeatedly performed.

The shear wave elastography measuring unit 40 measures the propagation velocity Vs of the shear wave with the measurement sequence explained using FIG. 3. The shear wave elastography measuring unit 40 calculates the propagation velocity Vs of the shear wave by depth in the subject. With this configuration, it is possible to obtain a measured value string including plural propagation velocities Vs corresponding to the plural depths. Further, in the shear wave measurement, the measurement sequence explained using FIG. 3 is performed plural times, thus a measurement set including the plural times of measurement sequence is performed, and plural measured value strings corresponding to the plural times of measurement sequence are obtained.

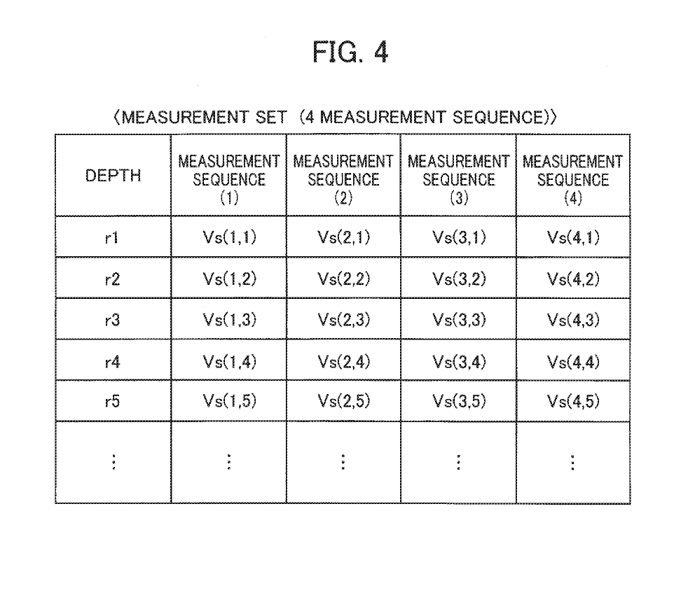

FIG. 4 is a diagram showing a concrete example of a measurement result at the propagation velocity Vs. FIG. 4 shows a measured value string of the propagation velocity Vs obtained by four times of measurement sequence. In the concrete example shown in FIG. 4, e.g., with the first measurement sequence (1), a measured value string including plural propagation velocities Vs(1,1), Vs(1,2), . . . corresponding to plural depths r1, r2, . . . are obtained; and with the second measurement sequence (2), a measured value string including plural propagation velocities Vs(2,1), Vs(2,2), . . . corresponding to the plural depths r1, r2, . . . are obtained. It goes without saying that a measurement set including more than five times or three or less times of measurement sequence may be performed.

When the measurement set including plural times of measurement sequence is performed and plural measured values (plural propagation velocities Vs) forming the measurement set are calculated, the shear wave elastography measuring unit 40 specifies at least one measured value which satisfies a rejection condition from the plural measured values. As the rejection condition, e.g. a condition based on magnitude of measured value (propagation velocity Vs), a condition based on tissue status in the subject, or the like may be preferable.

The shear wave elastography measuring unit 40 determines a propagation velocity Vs which satisfies the rejection condition as the object of rejection, from the calculated propagation velocities Vs, e.g. the plural propagation velocities Vs in the measurement set shown in FIG. 4. Note that the propagation velocity Vs determined as the object of rejection may be deleted from e.g. the measurement set shown in FIG. 4. Otherwise, it may be configured such that the value (data) of the propagation velocity Vs is not deleted, but a flag or the like, indicating that the value is the object of rejection, is set in correspondence with the value.

Then the shear wave elastography measuring unit 40 rejects the propagation velocity Vs which satisfies the rejection condition from the plural propagation velocities Vs in the measurement set. Then the shear wave elastography measuring unit 40 calculates the percentage of the plural propagation velocities Vs which have not been rejected but have remained, i.e., the plural propagation velocities Vs regarded as effective measured values, VsN (effective Vs percentage).

The shear wave elastography measuring unit 40 calculates the VsN by measurement sequence in the measurement set. For example, in the measurement set shown in FIG. 4, regarding the propagation velocities Vs of the plural depths forming the respective measurement sequences from the measurement sequence (1) to the measurement sequence (4), the shear wave elastography measuring unit 40 calculates the VsN (effective Vs percentage) by measurement sequence. Then, it may be configured such that e.g., when the VsN of respective measurement sequences is equal to or less than a threshold value, it is regarded that the reliability of the measurement sequence is low, and the propagation velocities Vs of all the depths in the measurement sequence are rejected. For example, in the concrete example in FIG. 4, when the VsN of the measurement sequence (3) is equal to or lower than 30% as the threshold value, all the propagation velocities Vs(3,1), Vs(3,2), . . . in the measurement sequence (3) are rejected.

Further, the shear wave elastography measuring unit 40 calculates statistical values related to the propagation velocities Vs, based on the plural propagation velocities Vs which have not been rejected but have remained i.e. plural propagation velocities regarded as effective measured values, among the plural propagation velocities Vs in the measurement set. As the statistical values, e.g., a mean value, a median value, an IQR, standard deviation, and a VsN (effective Vs percentage) related to the plural propagation velocities Vs regarded as effective measured values are preferable. Further, other statistical values may be calculated.

The ultrasonic diagnostic device in FIG. 1 has a function of combinational elastography for performing both of the strain elastography measurement and shear wave elastography measurement on the same diagnostic object. In the combinational elastography, when one measurement has been performed on a tissue in the subject, then another measurement is immediately performed on the tissue. For example, with respect to the tissue in the subject, when the strain elastography measurement sequence explained using FIG. 2(B) is performed, then the shear wave elastography measurement sequence explained using FIG. 3(B) is performed on the same tissue. For example, after strain elastography measurement, shear wave elastography measurement is performed in correspondence with switching operation from the user.

In the combinational elastography, the strain elastography measuring unit 30 performs the strain elastography measurement based on data in the first region of interest, and the shear wave elastography measuring unit 40 performs the shear wave elastography measurement based on data in the second region of interest. The first region of interest and the second region of interest are set with the region of interest setting unit 50 in correspondence with a user operation obtained by utilizing the operation unit 90. The user adjusts the positional relationship or the like between the first region of interest and the second region of interest, while watching e.g. a display image displayed on the display unit 82.

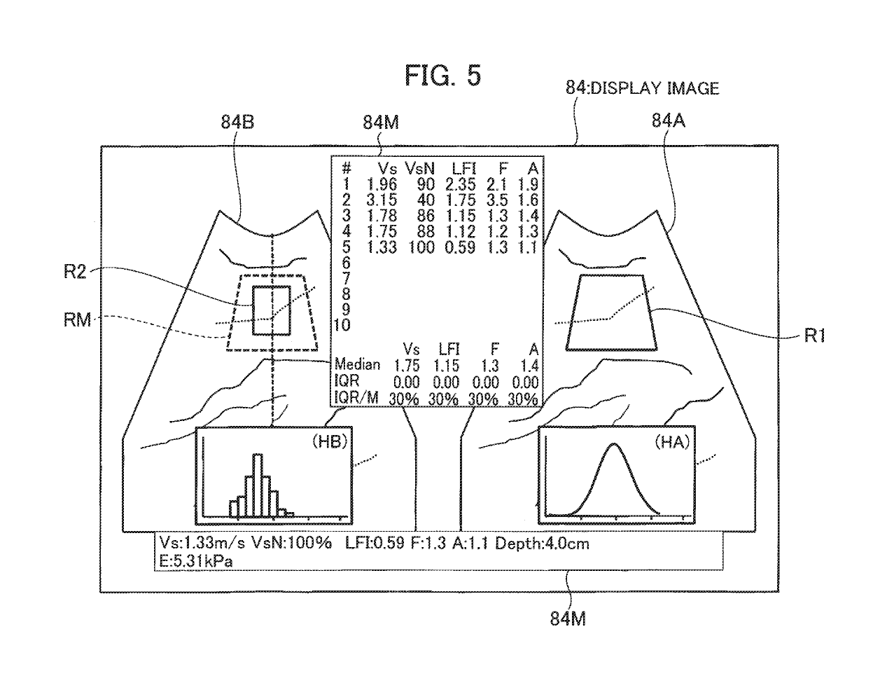

FIG. 5 is a diagram showing a concrete example of the display image in the combinational elastography. FIG. 5 shows a concrete example of a display image 84 formed with the display image forming unit 80 and displayed on the display unit 82. In the concrete example shown in FIG. 5, the display image 84 includes a strain elastography image 84A and a shear wave elastography image 84B.

The strain elastography image 84A is an image, indicating the elasticity information obtained from the strain elastography measurement with the strain elastography measuring unit 30, on a tomographic image (B mode image) formed with the tomographic image forming unit 20. A first marker R1 corresponding to the first region of interest is formed in the strain elastography image 84A. That is, the first marker R1 indicating the position, the shape and the size of the first region of interest is formed in the strain elastography image 84A. In the concrete example shown in FIG. 5, the first region of interest (first marker R1) has a trapezoidal shape but it may have any other shape such as a rectangular shape.

The strain elastography measuring unit 30 performs the strain elastography (strain elastography) based on the frame data in the first region of interest. With this configuration, an elasticity image representing the elasticity information at the plural measurement points in the first region of interest in color is formed. That is, in FIG. 5, in the first marker R1, colors corresponding to the elasticity information at each measurement point are added in the first marker R1.

The shear wave elastography image 84B is an image, showing a second marker R2 corresponding to the second region of interest utilized in the shear wave elastography measurement with the shear wave elastography measuring unit 40, on the tomographic image (B mode image) formed with the tomographic image forming unit 20. That is, the second marker R2 showing the position, the shape, and the size of the second region of interest is formed in the shear wave elastography image 84B. In the concrete example shown in FIG. 5, the second region of interest (second marker R2) has a rectangular shape but it may have any other shape.

Further, in the concrete example shown in FIG. 5, a reference marker RM corresponding to the first region of interest is formed in the shear wave elastography image 84B. That is, the reference marker RM indicating the position, the shape, and the size of the first region of interest is formed in the shear wave elastography image 84B.

In the combinational elastography, e.g., when the strain elastography measurement sequence (FIG. 2) has been performed on a tissue in the subject, the shear wave elastography measurement sequence (FIG. 3) is performed on the same tissue. Note that before the combinational elastography, the user such as a doctor or a laboratory technologist appropriately adjusts the position and the posture of the probe 10 such that the tissue as a diagnostic object is projected in the tomographic image (B mode image) displayed on the display unit 82.

In the combinational elastography, the display image forming unit 80 forms the display image 84 where the shear wave elastography image 84B and the strain elastography image 84A are horizontally arranged. The display image 84 is displayed on the display unit 82. In the first-performed strain elastography measurement, the display image forming unit 80 forms the first marker R1 corresponding to the first region of interest in the strain elastography image 84A. Further, the display image forming unit 80 forms the second marker R2 corresponding to the second region of interest and the reference marker RM corresponding to the first region of interest in the shear wave elastography image 84B.

The first region of interest and the second region of interest are set with the region of interest setting unit 50 in correspondence with the user operation obtained by utilizing the operation unit 90. The user adjusts the positional relationship or the like between the first marker R1 and the second marker R2, while watching e.g. the display image 84 displayed on the display unit 82. With this configuration, the positional relationship between the first region of interest and the second region of interest is adjusted.

In the first-performed strain elastography measurement, as the first marker R1 corresponding to the first region of interest and the second marker R2 corresponding to the second region of interest are formed in the display image 84, it is possible for the user to adjust the positional relationship or the like between the first region of interest and the second region of interest before e.g. the second-performed shear wave elastography, thus perform optimization. It is desirable that the adjustment is performed before determination of the measurement result of the first-performed strain elastography. Before the determination of the measurement result of the strain elastography, it is possible to optimize the position or the like of the first region of interest in the strain elastography, in consideration of even the second-performed second region of interest in the shear wave elastography.

Especially, by forming the reference marker RM together with the second market R2 in the shear wave elastography image 84B, the relative positional relationship between the first region of interest and the second region of interest is further clear. Note that it may be configured such that an unshown reference marker corresponding to the second region of interest is formed, together with the first marker R1, in the strain elastography image 84A. Note that not to disturb display of the elasticity image representing the elasticity information in the plural measurement points in colors in the first region of interest, it is desirable that it is possible to turn off (hide) the reference marker in the strain elastography image 84A.

Further, it is desirable that the region of interest setting unit 50 has a function of interlocking setting of changing the set position of one of the first region of interest and the second region of interest in accordance with change of the set position of the other one. In the interlocking setting, while maintaining e.g., the relative positional relationship between the first region of interest and the second region of interest, the one set position is changed in accordance with change of the other set position. Further, it is desirable that the region of interest setting unit 50 also has a function of individual setting of individually setting the first region of interest and the second region of interest. With the individual setting, it is possible to adjust the relative positional relationship between the first region of interest and the second region of interest.

In the combinational elastography, the shear wave elastography measurement, which is performed after the strain elastography, is performed in the second region of interest. For example, the pushing wave transmission beam P (FIG. 3) is formed so as to pass through the position P in the second region of interest. Further, the tracking wave ultrasonic beams T1 and T2 (FIG. 3) are formed so as to pass through the second region of interest. With this configuration, the propagation velocities Vs in plural depths in the second region of interest are measured.

Then, after the combinational elastography measurement, i.e., after the strain elastography measurement and the shear wave elastography measurement, a measurement result image 84M may be formed. In the measurement result image 84M, measured values obtained by the strain elastography and measured values obtained by the shear wave elastography are displayed as e.g. numerical values.

Further, a histogram HA based on the elasticity information obtained by the strain elastography measurement may be formed. In the concrete example shown in FIG. 5, the histogram HA is formed on the strain elastography image 84A. In the histogram HA, a lateral axis indicates values of the elasticity information (strain and elastic modulus of a tissue), and a vertical axis indicates frequency. Note that the histogram HA may be formed based on the elasticity information in the first region of interest. Otherwise, the histogram HA may be formed based on the elasticity information in histogram region (window) set separately from the first region of interest.

Further, a histogram HB may be formed based on the measurement result obtained by the shear wave elastography measurement. In the concrete example shown in FIG. 5, the histogram HB is formed on the shear wave elastography image 84B. In the histogram HB, the lateral axis indicates a measurement result value (propagation velocity Vs), and the vertical axis, frequency.

According to the combinational elastography, it is possible to perform the shear wave elastography measurement immediately after the strain elastography measurement. Accordingly, it is possible to obtain e.g. the measurement result of the shear wave elastography and the measurement result of the strain elastography, in substantially the same cross section and the same respiratory period regarding the same tissue. Note that in the combinational elastography, it may be configured such that the shear wave elastography measurement is performed first and then the strain elastography measurement is performed.

In this manner, when the combinational elastography is performed, the diagnosis processing unit 60 derives a general diagnostic result regarding the tissue based on the measurement result obtained from the tissue in the subject by the strain elastography measurement and the measurement result obtained from the same tissue by the shear wave elastography measurement. Note that in the derivation of the general diagnostic result, it is desirable to refer to blood data obtained with the blood data acquisition unit 70.

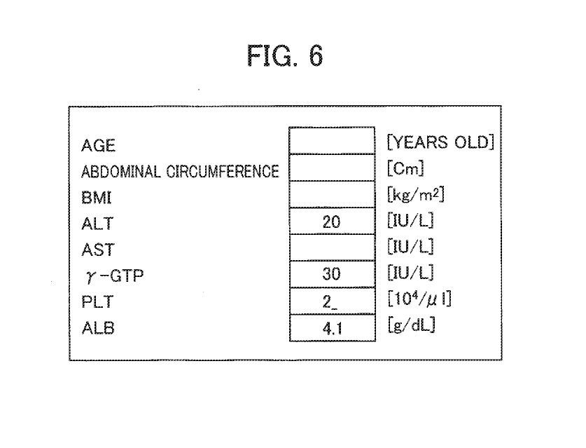

FIG. 6 is a diagram showing a concrete example of blood data. The blood data includes information on the subject's blood such as ALT, AST, γGTP and the like. It is obtained by e.g. inspection before the combinational elastography measurement with the ultrasonic diagnostic device in FIG. 1.

Then, e.g. a user interface screen to input blood data as shown in FIG. 6 is displayed on the display unit 82. The blood data is inputted by the user such as a doctor or a laboratory technologist, and the blood data acquisition unit 70 obtains the blood data. Note that it is desirable that it is possible to input subject information such as the age, the abdominal circumference, the BMI and the like of the subject, with the user interface screen to input blood data.

The diagnosis processing unit 60 calculates e.g. a fibrosis score F value and an inflammation score A value as indices corresponding to Fibrosis (fibrosis) and Activity (activity of inflammation) as factors defining the progress of hepatic fibrosis, as a general diagnostic result. The fibrosis score F value and the inflammation score A value are respectively calculated with e.g. expression 1 and expression 2.

F value=Vs×[F₁]+IQR×[F₂]+ . . . +strain mean value×[Fₘ]+strain standard deviation×[Fₘ₊₁]+ . . . +ALT×[Fₙ]+AST ×[Fₙ₊₁]+γGTP ×[Fₙ₊₂]+ . . . +age×[Fₕ]+abdominal circumference×[Fₕ₊₁]+BMI×[Fₕ₊₂]+ [Expression 1]

A value=Vs×[A₁]+IQR×[A₂]+ . . . +strain mean value×[Aₘ]+strain standard deviation×[Aₘ₊₁]+ . . . +ALT×[Aₙ]+AST×[Aₙ₊₁]+γGTP×[Aₙ₊₂]+ . . . +age×[Aₕ]+abdominal circumference×[Aₕ₊₁]+BMI×[Aₕ₊₂]+ [Expression 2]

The expression 1 and the expression 2 include “Vs”, “IQR” and the like obtained by the combinational elastography shear wave elastography measurement. The value “Vs” is a mean value related to e.g. the plural propagation velocities Vs regarded as effective measured values. The value “IQR” is an IQR (interquartile range) related to e.g. the plural propagation velocities Vs regarded as effective measured values.

Further, the expression 1 and the expression 2 include “strain mean value”, “strain standard deviation” and the like obtained by the combinational elastography strain elastography measurement. The values “strain mean value” and “strain standard deviation” are respectively, a mean value and standard deviation related to e.g. the elasticity information (strain and elastic modulus of a tissue) in plural measurement points in the first region of interest.

Further, the expression 1 and the expression 2 include values “ALT”, “AST”, “γGTP” and the like as blood data of the subject as the object of the combinational elastography measurement. Further, the expression 1 and the expression 2 include subject information such as the age, the abdominal circumference, and the BMI value of the subject as the object of the combinational elastography measurement.

Note that [F₁], [F₂], [Fₘ], [Fₘ₊₁], [Fₙ], [Fₙ₊₁], [Fₙ₊₂], [Fₕ], [Fₕ₊₁], [Fₕ₊₂] . . . in the expression 1 are coefficients to obtain the fibrosis score F value. Further, [A₁], [A₂], [Aₘ], [Aₘ₊₁], [Aₙ], [Aₙ₊₁], [Aₙ₊₂], [Aₕ], [Aₕ₊₁], [Aₕ₊₂] . . . in the expression 2 are coefficients to obtain the inflammation score A value.

The diagnosis processing unit 60 calculates the fibrosis score F value and the inflammation score A value utilizing e.g. the expression 1 and the expression 2. The expression 1 and the expression 2 include, in addition to the measurement results obtained by the combinational elastography (shear wave elastography measurement and the strain elastography measurement), the items related to the blood data and the items related to the subject information. Accordingly, improvement of diagnostic accuracy is expected in comparison with a case where blood data and subject information are not utilized. Note that it may be configured such that the fibrosis score F value and the inflammation score A value are calculated with, e.g. the expression 1 and the expression 2, in which the items related to the blood data and the items related to the subject information are omitted, only utilizing the measurement results obtained from the combinational elastography shear wave elastography measurement and the strain elastography measurement. Further, in the expression 1 and the expression 2, any one of the items related to the blood data and the items related to the subject information may be omitted.

As described above, the preferred embodiment of the present invention has been described, however, the above embodiment is merely an example in every point, but does not limit the scope of the present invention. The present invention includes various modifications without departing from the subject matter of the invention.

### LIST OF REFERENCE SIGNS

10 . . . probe, 12 . . . transmission unit, 14 . . . reception unit, 20 . . . tomographic image forming unit, 30 . . . strain elastography measuring unit, 40 . . . shear wave elastography measuring unit, 50 . . . region of interest setting unit, 60 . . . diagnosis processing unit, 70 . . . blood data acquisition unit, 80 . . . display image forming unit, 82 . . . display unit, 100 . . . control unit.

1. An ultrasonic diagnostic device comprising:
an elasticity measurement unit that performs elasticity measurement according to a first method and elasticity measurement according to a second method based on data obtained by transmission/reception of an ultrasonic wave;
a region of interest setting unit that sets a first region of interest corresponding to the elasticity measurement according to the first method and a second region of interest corresponding to the elasticity measurement according to the second method; and
a display image forming unit that forms a first display image corresponding to the elasticity measurement according to the first method and a second display image corresponding to the elasticity measurement according to the second method,
wherein the elasticity measurement unit performs the elasticity measurement according to the first method based on data in the first region of interest in the elasticity measurement according to the first method, and
wherein the display image forming unit forms a display image including the first display image and the second display image, forms a first marker corresponding to the first region of interest in the first display image, and forms a second marker corresponding to the second region of interest in the second display image, in the elasticity measurement according to the first method,
further wherein the elasticity measurement unit performs the elasticity measurement according to the second method based on data in the second region of interest in the elasticity measurement according to the second method after the elasticity measurement according to the first method.

2. The ultrasonic diagnostic device according to claim 1,
wherein the display image forming unit forms a reference marker corresponding to the first region of interest, together with the second marker, in the second display image, in the elasticity measurement according to the first method.

3. The ultrasonic diagnostic device according to claim 1,
wherein the region of interest setting unit has an interlocking setting function of changing a setting position of one of the first region of interest and the second region of interest in accordance with change of the setting position of the other one.

4. The ultrasonic diagnostic device according to claim 2,
wherein the region of interest setting unit has an interlocking setting function of changing a setting position of one of the first region of interest and the second region of interest in accordance with change of the setting position of the other one.

5. The ultrasonic diagnostic device according to claim 1,
wherein the elasticity measurement unit performs strain elastography measurement based on displacement distribution of a tissue in a living body, as the elasticity measurement according to the first method, and performs shear wave elastography measurement based on a shear wave propagated through the tissue in the living body, as the elasticity measurement according to the second method.

6. The ultrasonic diagnostic device according to claim 2,
wherein the elasticity measurement unit performs strain elastography measurement based on displacement distribution of a tissue in a living body, as the elasticity measurement according to the first method, and performs shear wave elastography measurement based on a shear wave propagated through the tissue in the living body, as the elasticity measurement according to the second method.

7. The ultrasonic diagnostic device according to claim 3,
wherein the elasticity measurement unit performs strain elastography measurement based on displacement distribution of a tissue in a living body, as the elasticity measurement according to the first method, and performs shear wave elastography measurement based on a shear wave propagated through the tissue in the living body, as the elasticity measurement according to the second method.

8. The ultrasonic diagnostic device according to claim 5,
wherein the device obtains a general diagnostic result of the tissue in the living body based on a measurement result obtained from the tissue in the living body by the strain elastography measurement and a measurement result obtained from the tissue in the living body by the shear wave elastography measurement.

9. The ultrasonic diagnostic device according to claim 6,
wherein the device obtains a general diagnostic result of the tissue in the living body based on a measurement result obtained from the tissue in the living body by the strain elastography measurement and a measurement result obtained from the tissue in the living body by the shear wave elastography measurement.

10. The ultrasonic diagnostic device according to claim 7,
wherein the device obtains a general diagnostic result of the tissue in the living body based on a measurement result obtained from the tissue in the living body by the strain elastography measurement and a measurement result obtained from the tissue in the living body by the shear wave elastography measurement.

11. The ultrasonic diagnostic device according to claim 5,
wherein the device obtains a general diagnostic result of the tissue in the living body based on a measurement result obtained from the tissue in the living body by the strain elastography measurement, a measurement result obtained from the tissue in the living body by the shear wave elastography measurement, and blood data of the subject having the tissue in the living body.

12. The ultrasonic diagnostic device according to claim 6,
wherein the device obtains a general diagnostic result of the tissue in the living body based on a measurement result obtained from the tissue in the living body by the strain elastography measurement, a measurement result obtained from the tissue in the living body by the shear wave elastography measurement, and blood data of the subject having the tissue in the living body.

13. The ultrasonic diagnostic device according to claim 7,
wherein the device obtains a general diagnostic result of the tissue in the living body based on a measurement result obtained from the tissue in the living body by the strain elastography measurement, a measurement result obtained from the tissue in the living body by the shear wave elastography measurement, and blood data of the subject having the tissue in the living body.
